(19) United States
(12) Patent Application Publication
(10) Pub. No.: US 2011/0270241 A1
(43) Pub. Date: Nov. 3, 2011

(54) VISUAL FEEDBACK IMPLEMENTS FOR ELECTROMAGNETIC ENERGY OUTPUT DEVICES

(75) Inventor: Dmitri Boutoussov, Dana Point, CA (US)

(73) Assignee: BIOLASE TECHNOLOGY, INC.

(21) Appl. No.: 13/180,441

(22) Filed: Jul. 11, 2011

Related U.S. Application Data
(63) Continuation of application No. 11/475,719, filed on Jun. 26, 2006.
(60) Provisional application No. 60/693,705, filed on Jun. 24, 2005.
(60) Provisional application No. 60/700,510, filed on Jul. 18, 2005.
(60) Provisional application No. 60/751,076, filed on Dec. 15, 2005.

(51) Int. Cl. A61B 18/18

(52) U.S. Cl. 606/33

(57) ABSTRACT

An electromagnetic energy output device in the form of laser handpiece and a trunk assembly is disclosed. The electromagnetic energy output device includes a digital camera and electromagnetic energy waveguides for emitting illumination or excitation light energy to enhance user viewability of a target surface and signal analysis and to receive electromagnetic energy such as return excitation light. An image acquisition fitting routes images acquired at or in a vicinity of the distal end of the electromagnetic energy output device. The image acquisition fitting can include an attachable or clip-on element or set of elements. In other implementations, the image acquisition fitting may be securable, in whole or in part, within an interior of the electromagnetic energy output device.

### CROSS-REFERENCE TO RELATED APPLICATIONS

This application is a continuation of co-pending U.S. application Ser. No. 11/475,719, filed Jun. 26, 2006 and entitled VISUAL FEEDBACK IMPLEMENTS FOR ELECTROMAGNETIC ENERGY OUTPUT DEVICES (Att. Docket BI9936P), which claims the benefit of U.S. Provisional Application No. 60/693,705, filed Jun. 24, 2005 and entitled. ELECTROMAGNETIC ENERGY OUTPUT DEVICES HAVING VISUAL FEEDBACK FOR DENTAL AND OTHER APPLICATIONS (Att. Docket BI9901PR), U.S. Provisional Application No. 60/700,510, filed Jul. 18, 2005 and entitled ELECTROMAGNETIC ENERGY OUTPUT DEVICES HAVING VISUAL FEEDBACK FOR DENTAL AND OTHER APPLICATIONS (Att. Docket BI9901PR2), and U.S. Provisional Application No. 60/751,076, filed Dec. 15, 2005 and entitled VISUAL FEEDBACK IMPLEMENTS FOR ELECTROMAGNETIC ENERGY OUTPUT DEVICES (Att. Docket BI9936PR), the entire contents of all which are hereby incorporated by reference.

### BACKGROUND OF THE INVENTION

1. Field of the Invention

The present invention relates to electromagnetic energy output devices and, more particularly, to visual feedback implements for use with medical procedure handpieces that output electromagnetic energy and to implements and methods for using these items,

2. Description of Related Art

A wide variety of electromagnetic energy output devices and visualization implements have existed in the prior art, including laser hand pieces for performing or facilitating the performance of medical procedures and also including medical-use cameras for providing visual feedback to a user engaged in the performance of a medical operation such as a laparoscopic process.

### SUMMARY OF THE INVENTION

An electromagnetic energy output device according to the present invention comprises a laser handpiece and a trunk assembly. The electromagnetic energy output device includes a digital camera and electromagnetic energy waveguides for emitting illumination or excitation light energy to enhance user viewability of a target surface and signal analysis and for receiving electromagnetic energy such as return excitation light. An image acquisition fitting routes images acquired at or in a vicinity of the distal end of the electromagnetic energy output device.

In accordance with certain implementations of the present invention, an electromagnetic energy output device is provided in the form of a handpiece (e.g., a laser handpiece such as a dental handpiece) and a trunk assembly that may comprise, for example, an image-acquisition device such as a digital camera and one or more electromagnetic energy waveguides for emitting electromagnetic energy, such as illumination or excitation light for purposes of enhancing user viewability of a target surface or signal analysis, and for receiving electromagnetic energy such as return excitation light. The assembly of items can further comprise an image acquisition fitting for performing one or more of various functions, such as routing images (e.g., working-surface images) acquired at or in a vicinity of the distal end of the electromagnetic energy output device. The image acquisition fitting may be autoclavable, in whole or in part, and may comprise, for example, an attachable or clip-on element or set of elements. In other implementations, the image acquisition fitting may be securable, in whole or in part, within an interior of the electromagnetic energy output device.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112.

Any feature or combination of features described herein are included within the scope of the present invention provided that the features included in any such combination are not mutually inconsistent as will be apparent from the context, this specification, and the knowledge of one skilled in the art. In addition, any feature or combination of features may be specifically excluded from any embodiment of the present invention. For purposes of summarizing the present invention, certain aspects, advantages and novel features of the present invention are described. Of course, it is to be understood that not necessarily all such aspects, advantages or features will be embodied in any particular implementation of the present invention. Additional advantages and aspects of the present invention are apparent in the following detailed description and claims that follow.

### BRIEF DESCRIPTION OF THE DRAWINGS

FIG. 1 is a side-elevation diagram of an embodiment of a device having imaging structure and being constructed for treating a target area of tissue;

FIG. 2 is a schematic representation of one implementation of a rod lens assembly for use with the device of FIG. 1;

FIG. 3A illustrates in schematic form a construction of the rod lens assembly such as that usable with FIGS. 1 and 2;

FIG. 3B shows an internal-mount construction corresponding to the architecture of FIG. 3A which internal-mount construction can be formed with or without a rod lens assembly;

FIGS. 3B(1) to 3B(4) illustrate various configurations of embodiments in which a beam-bending element is used to alter a direction of an optical pathway from an optical axis of a visual feedback implement to an optical axis of an imaging fiber;

FIG. 4 depicts a laser handpiece and a trunk assembly having an image-acquisition device and one or more electromagnetic energy waveguides for emitting illumination or excitation light energy to enhance user viewability of a target surface or to facilitate signal analysis;

FIG. 5A shows a cross-sectional or end view of a point along the optical path distal of the visual feedback implement, wherein the view includes an image-acquisition device disposed in a central lumen with an illumination/excitation light-source waveguide and a return-tight waveguide disposed external to the central lumen;

FIG. 5B shows a cross-sectional or end view of a point along the optical path distal of the visual feedback implement, wherein the image-acquisition device is again disposed in a central lumen with an illumination/excitation light-source waveguide disposed concentrically around the central lumen and with a first return-light waveguide and a second return-light waveguide disposed external to the light-source waveguide;

FIGS. 6A-6D depict variations of an imaging fiber such as that which can be constructed and used in conjunction with the assembly of FIG. 4; and

FIG. 7 is a block diagram of a portion of an exemplary apparatus that may be used to detect dental caries according to an implementation of the present invention.

### DETAILED DESCRIPTION OF THE INVENTION

Reference will now be made in detail to the presently preferred embodiments of the invention, examples of which are illustrated in accompanying drawings. Wherever possible, the same or similar reference numbers are used in drawings and the description to refer to the same or like parts. It should be noted that any drawings presented are in simplified form and are not to precise scale. In reference to the disclosure herein, for purposes of convenience and clarity only, directional terms, such as, top, bottom, right, up, down, over, above, below, beneath, rear, and front, are used with respect to the accompanying drawings. Such directional terms should not be construed to limit the scope of the invention in any manner.

Although the disclosure herein refers to certain illustrated embodiments, it is to be understood that these embodiments are presented by way of example and not by way of limitation. The intent of the following detailed description, although discussing exemplary embodiments, is to be construed to cover all modifications, alternatives, and equivalents of the embodiments as may fall within the spirit and scope of the invention as defined by the appended claims. It is to be understood and appreciated that the process steps and structures described or incorporated by reference herein do not cover a complete process flow for the implementations described herein. The present invention may be practiced in conjunction with various methods and devices that are conventionally used in the art, and only so much of the commonly practiced method steps and structures are included herein as are necessary to provide an understanding of the present invention.

Any feature or combination of features described herein are included within the scope of the present invention provided that the features included in any such combination are not mutually inconsistent as will be apparent from the context, this specification, and the knowledge of one of ordinary skill in the art.

The present invention relates to visual feedback implements for being coupled and operated with medical procedure handpieces and implements and to methods for using these items. This invention contemplates constructions and uses of visual feedback implements cameras) as described in, for example, U.S. application Ser. No. 11/441,788, filed May 25, 2006 and entitled DEVICE HAVING ACTIVATED TEXTURED SURFACES FOR TREATING ORAL TISSUE (Att. Docket BI9878P), and U.S. application Ser. No. 11/413,590, filed Apr. 26, 2006 and entitled METHODS FOR TREATING EYE CONDITIONS (Att. Docket BI9852P; now U.S. Pat. No. 7,665,467), the entire contents of both which are incorporated herein by reference. A few medical (e.g., dental) applications for the presently-described visual-feedback treatment devices, in addition to those described or suggested herein and in documents referenced herein, may include periodontal pockets (e.g., diagnostic and treatment), endodontics (e.g., visualization of canals), micro-dentistry, tunnel preparations, caries detection and treatment, bacteria visualization and treatment, general dentistry, and airborne-agent and gas detection applications as described in U.S. Provisional Application No. 60/739,314.

With reference to the drawings, FIG. 1 shows an electromagnetic energy output device 10 which can be configured to perform a medical (e.g., dental) procedure. The electromagnetic energy output device 10 may take the form of a laser handpiece having an output fiber tip 12, such as a periodontal tip, for emitting laser energy toward a target surface. In the illustrated embodiment, the electromagnetic energy output device 10 is coupled with imaging structure 14 that may terminate near a distal end 18 of the electromagnetic energy output device 10 at a point 11 or that may terminate with an imaging fiber 16. In implementations comprising an imaging fiber, the imaging fiber 16 may extend, for example, to a vicinity of, or to the end of the output fiber tip 12. According to certain implementations, the imaging fiber 16 may be held in a vicinity of, or may be secured to, the output fiber tip 12. In embodiments comprising imaging fibers 16 that are secured to the output fiber tip 12, such as imaging fibers with output ends that terminate at the output end of the output fiber tip (e.g., are coterminous with the output end of the output fiber tip), the imaging fiber or fibers may be attached to the output fiber tip (e.g., to form a single assembly) with, for example, a band or with a bonding material such as an adhesive.

In certain embodiments, one or more optical fibers (e.g., forming a coherent fiber bundle) can be provided that are configured to transmit light via, for example, a waveguide 17 such as of a rod lens assembly 15, from the distal end 18 to a proximal end 20 of the electromagnetic energy output device 10, for routing images (e.g., working-surface images) acquired at or in a vicinity of the distal end 18 by a visual feedback implement 22. The one or more optical fibers can additionally or alternatively be configured to transmit light via, for example, the same or a different waveguide 17 (e.g., of the rod lens assembly 15), from the proximal end 20 to the distal end 18 of the electromagnetic energy output device 10. According to some embodiments, the visual feedback implement 22 can comprise an image-acquisition device (e.g., CCD or CMOS cameras) for obtaining or processing light information (e.g., images) from the distal end 18. In the illustrated embodiment, the visual feedback implement 22 comprises a camera chip with a camera interface (e.g., which may comprise a focusing element and/or a zoom lens).

According to this and any of the other embodiments described or referenced herein, one or more of the optical fibers or other conduits (e.g., air and water lines) can be arranged, for example, outside of the electromagnetic energy output device 10 (e.g., laser handpiece) envelope such as in the form of an attachment (e,g., removable attachment) as shown in FIGS, 1 and 4, or can be arranged either partially or completely inside (e.g., internally mounted) of the electromagnetic energy output device 10 envelope.

The visual feedback implements disclosed herein and in the above-referenced applications can be disposed on (e.g., removably attached, such as to output ends of) or in a vicinity of (e.g., not attached to output ends of) electromagnetic energy output devices (e.g., lasers and dental lasers, such as handpieces), to thereby form visual-feedback treatment devices in accordance with the present invention. The visual feedback implement can thus be built-in or removably attached to a handpiece and, further, can be disposed at any of a variety of locations on or in connection with the handpiece between the proximal end and distal end, or proximally of the proximal end. In particular instances, the visual feedback implements can be used, for example, (a) in a form integrated in a handpiece or output end of an electromagnetic energy output device, (b) in a form attached to the handpiece or electromagnetic energy output device, or (c) in conjunction with (e.g., not necessarily attached to) the handpiece or electromagnetic energy output device, wherein such handpieces and devices can facilitate, for example, information collection, diagnostics, cutting, ablating, treatments, and the like.

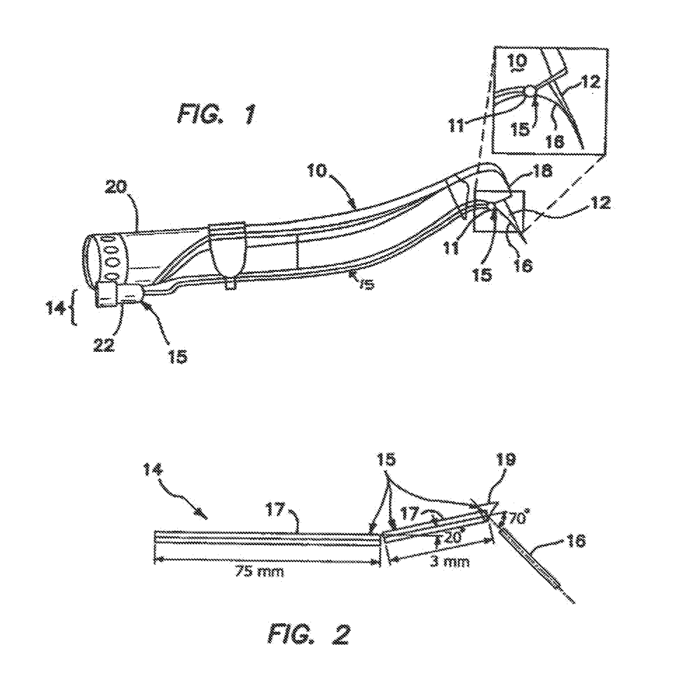

A schematic representation of one implementation of the rod tens assembly 15 of FIG. 1 is shown in FIG, 2. In the exemplary embodiment of FIG. 2, the rod lens assembly 15 is formed as a removable attachment which can be affixed to an exterior surface of a handpiece and which may comprise, for example, a 75 mm rod and a 35 mm rod that terminates at its distal end with a beam bending element 19 (e.g., prism) for forming an optical pathway to the output fiber tip 12.

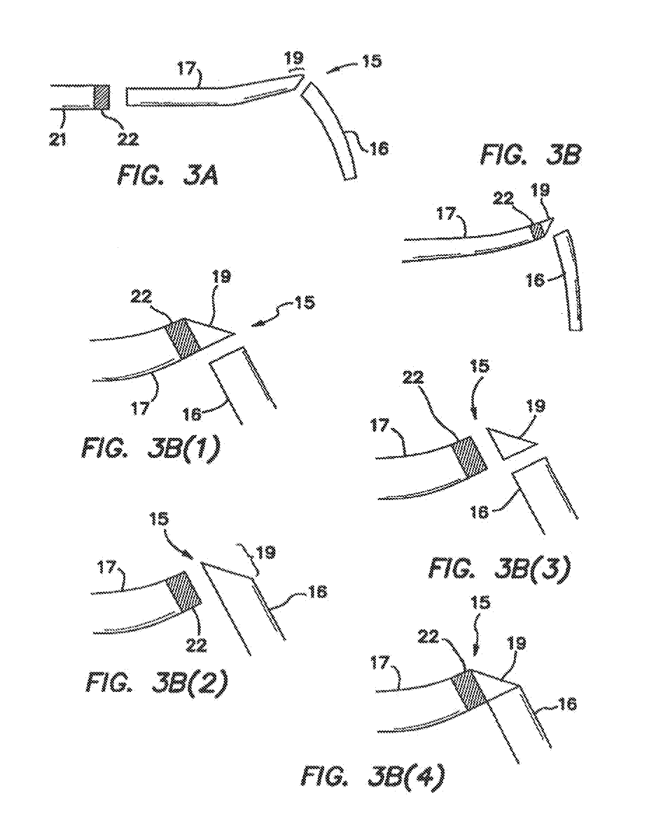

With reference to FIG. 3A, a construction of the rod lens assembly 15 from FIGS. 1 and 2 is shown in schematic form wherein a diameter of the externally-mounted (e.g., on an exterior of the handpiece) rod lens assembly 15 is about 1.5 mm and a proximal end of the rod lens assembly is coupled to a visual feedback implement 22 such as a ¼″ camera. The visual feedback implement 22 (e.g., camera) can, in turn, be coupled to a waveguide 21 which extends proximally of the camera 22, and a distal end of the rod tens assembly 15 may (1) terminate with or without the addition of interface optics for emitting light from the rod lens assembly and/or receiving tight from the target, or (2) comprise or be coupled to, with or without the addition of interface optics and with or without a beam bending element 19 for altering a direction of the optical pathway, an imaging fiber 16 (e.g., a removable imaging fiber).

Example operation specifications for the embodiment of FIG. 3A are provided in TABLE 1 and TABLE 2 below.

TABLE 1
Optical Parameters for Combination of Camera and Rod Lens Assembly

| | |
|---|---|
| Field of View | 30-45 degrees |
| Viewing Angle | ~70 degrees |
| Resolution | 50 micrometers |
| Focal Range | 4-15 mm |
| Outer Diameter | max 2.5 mm |
| Illumination | LED, white Luxeon, _ mcd |

TABLE 2
Optical Parameters for imaging Fiber Only

| | |
|---|---|
| Length | 12-14 mm |
| Field of View | 45-90 degrees |
| Viewing Angle | 0 degrees |
| Resolution | 20 micrometers |
| Focal Range | 0-2 mm |
| Outer Diameter | Max 1.0 mm |
| Illumination | LED, white Luxeon, _ mcd |

In FIG. 3B, an internal-mount construction corresponding to the architecture of FIG. 3A can be formed with or without a rod lens assembly. According to the illustrated embodiment, the internal-mount construction is formed without a rod lens assembly. The construction of FIG. 3B can be formed, as presently embodied, with a visual feedback implement, which may take the form of, for example, a camera 22, and which, alone or in combination with one or more other components, may be reduced in size in accordance with certain implementations to, for example, facilitate internal-mounting within the handpiece. The visual feedback implement 22 (e.g., reduced-size camera) may comprise, for example, a 1 mm camera having a proximal end coupled to a waveguide 17 that extends proximally of the camera. 22 and having a distal end that may comprise or be coupled to a beam bending element 19. As with the implementation of FIG. 3A, a distal end of the camera 22 may (1) terminate with or without the addition of interface optics for emitting light from the camera 22 and/or receiving light from the target, or (2) comprise or be coupled to, with or without the addition of interface optics and with or without a beam bending element 19 for altering a direction of the optical pathway, an imaging fiber 16 (e.g., a removable imaging fiber).

FIGS. 3B(1) to 313(4) illustrate various configurations of embodiments in which a beam-bending element 19 in the form of, for example, a prism, is used to alter a direction of the optical pathway from an optical axis of the visual feedback implement 22 (e.g., a camera chip) to an optical axis of the imaging fiber 16 (e.g., a removable imaging fiber).

Termination of the distal end of the rod lens assembly 15 or camera 22 without an imaging fiber 16 (with or without interface optics), can facilitate, for example, a general view of the target surface. For example, such a termination may be made with interface optics that may operate to provide the functionality of, for example, a relatively wide-angle view camera, thus allowing for example an operator of a handpiece to observe a tooth or gum surface. In other embodiments, zero- or zoom-magnification optics or technology may be implemented to provide a narrower-angle view and/or zoom-in and zoom-out capabilities.

On the other hand, termination of the distal end of the rod lens assembly 15 or camera 22 with an imaging fiber 16 (with or without interface optics can facilitate, for example, a more specialized (e.g., close-up) view of one or more particular aspects of the target surface. For example, the imaging fiber, which may terminate at the output surface of the output fiber tip, may operate to provide the functionality of a target-close mounted camera, thus allowing for example an operator of a handpiece to observe, for example, interior surfaces of various sites (e.g., under tissue-surface sites) such as a periodontal pocket.

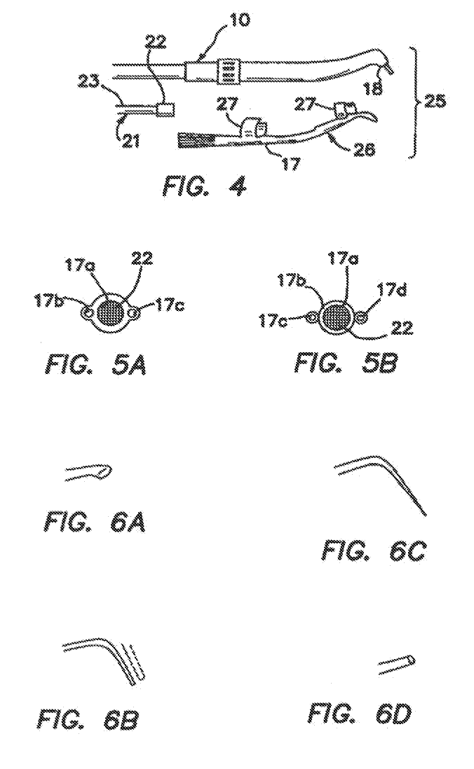

With reference to FIG. 4, an assembly of items is shown comprising an electromagnetic energy output device 10 in the form of a handpiece (e.g., a laser handpiece such as a dental handpiece) and a trunk assembly 21 that may comprise, for example, an image-acquisition &vice 22 such as a digital camera and/or one or more electromagnetic energy waveguides 23 for emitting electromagnetic energy (e.g., such as illumination or excitation light energy for purposes of enhancing user viewability of a target surface or signal analysis) and/or receiving electromagnetic energy (e.g., such as return excitation light).

The assembly of items 25 shown in FIG, 4 can further comprise an image acquisition fitting 26 for performing one or more of various functions, such as routing images (e.g., working-surface images) acquired at or in a vicinity of the distal end 18 of the electromagnetic energy output device 10. The image acquisition fitting 26 may be autoclavable, in whole or in part, and may comprise, for example, an attachable or clip-on element or set of elements 27. In other implementations, the image acquisition fitting 26 may be securable, in whole or in part, within an interior of the electromagnetic energy output device 10. According to the illustrated embodiments, the image acquisition fitting 26 may take the form or functionality, in whole or in part, of any one or more of the implementations and interconnections shown in FIGS. 3A and 3B.

Any two or more elements of the rod tens assembly 15, beam bending element 19 and imaging fiber 16, to the extent included, of the exemplary assembly of FIG. 3A, or of any variation thereof, may be fabricated or assembled as part of a single structure (e.g., a single image acquisition fitting) wherein, furthermore, any two or more of the elements in any combination or permutation may be constructed (e.g., be formed or bonded together) to contact one another along an optical pathway of the single structure or may be formed physically isolated from one another. Similarly, any two or more elements of the waveguide 17, image acquisition device 22, beam bending element 19 and imaging fiber 16, to the extent included, of the exemplary assembly of FIG. 3B, or of any variation thereof, may be fabricated or assembled as part of a single structure (e.g., a single image acquisition fitting) wherein any two or more of the elements in any combination or permutation may be constructed (e.g., be formed or bonded together) to contact one another along an optical pathway of the single structure or may be formed to be physically isolated from one another.

In this regard, implementations wherein the image-acquisition device 22 is disposed within or in a vicinity of any location along a length of the trunk assembly 21 (e.g., near a distal end of the trunk assembly 21) can correspond, for example, to the content of FIG. 3A. Moreover, in other implementations of the assembly of items 25 shown in FIG. 4, the image-acquisition device 22 can be disposed within or in a vicinity of any location along a length of the image acquisition fitting 26 (e.g., near a proximal end of the image acquisition fitting 26 or, as another example of many examples, near a distal end of the image acquisition fitting 26) thereby corresponding, for example, in whole or in part, to the content of FIG. 3B.

Furthermore, any of the modifications that may be applied to the implementations of FIGS. 3A and 3B as discussed above, such as, for example, omission of the imaging fiber 16, may be incorporated into the various implementations of the assembly of items 25 described in connection with FIG. 4.

Still further, in addition to the incorporation of any of the modifications that may be applied to the implementations of FIGS. 3A and 3B as discussed above, variations of the imaging fiber 16, to the extent used, may be constructed such as depicted in FIGS. 6A-6D, for incorporation with the various embodiments of the assembly of items 25 described in conjunction with FIG. 4. Any of the imaging fiber 16 constructions depicted in FIGS. 6A-6D may be autoclavable, in whole or in part.

FIG. 6A shows an open space viewer of 90 degrees, and FIG. 6B shows a periodontal probe such as that depicted in FIG. 1 (the difference being that the FIG. 1 embodiment comprises an imaging fiber which may comprise a non-tapered construction) with a position of the output fiber tip being shown in phantom for reference. FIG. 6C shows an endodontal probe, which, as distinguished from the periodontal probe of FIG. 6B which may have a diameter of for example 1 mm, may have a diameter of for example about 0.2 mm and may comprise a more elongate and/or tapered structure, and FIG. 6D shows an open space viewer of 0 degrees.

Regarding particular applications of the visual feedback implement and electromagnetic energy (e.g., laser) system of the present invention, the visual-feedback treatment device can be used to provide, for example, real-time diagnostic and treatment information regarding tissues being accessed or treated (e.g., diagnosed, cut or ablated), before, during and after treatments. For example, during or after a step or sub-step has been performed during a diagnostic or treatment procedure, the site of operation can be viewed, in real time and/or without having to remove the visual-feedback treatment device (e.g., laser, in embodiments wherein the visual feedback implement is already attached near a distal end of a laser handpiece).

The visual-feedback treatment device of the present invention can be used, for instance, before, during and after procedures involving the diagnosing (e.g., of diseased or swollen tissues) and/or treating of targets, such as periodontal pockets. At any point prior to, during, or after, any step or sub-step of the procedure, real-time visual feedback of the operation site can be instantaneously obtained for automated processing (e.g., by hardware or software) and/or for observation/review/analysis by a user, and implementation of steps or sub-steps of the procedure can be generated or modified based upon the visual feedback.

Types of bacteria that, for example, can be either labeled and visualized under light or that produce a fluorescent emission that can be visualized through a camera are also applicable to this invention. Visualization of bacteria may be enhanced through addition of various markers that can be easily recognized by various wavelengths. The camera can also be equipped with a data input output analysis system that can track tissue repair and various aspects of treatment such as pocket reduction.

FIG. 5A shows a cross sectional or end view of a point along the optical path distal of the visual feedback implement 22 (e.g., camera), such as, for example, at an interface between the camera 22 and rod tens assembly 15 of the construction of FIG. 3A or near a distal end of the trunk assembly 21 of FIG. 4. In the cross-sectional view of FIG. 5A, an image-acquisition device 22 (e.g., CCD or CMOS camera) is disposed in a central lumen 17a, with an illumination/excitation light-source waveguide 17b and a return-tight waveguide 17c being disposed external to (e.g., on opposing sides of) the central lumen 17a. The illustrated embodiment shows the illumination/excitation light-source waveguide 7b emitting white light and the return-light waveguide 17c returning light that includes wavelengths of 405 nanometers and 635 nanometers.

The cross-sectional view of FIG. 5B shows the image-acquisition device 22 (e.g., CCD or CMOS camera again disposed in a central lumen 17a, with an illumination/excitation light-source waveguide 17b being disposed concentrically around the central lumen 17a and with a first return-light waveguide 17c and a second return-light waveguide 17d being disposed external to (e.g., on opposing sides of) the light-source waveguide 17b. This illustrated embodiment shows the illumination/excitation light-source waveguide 17b emitting white light and the first and second return-light waveguides 17b, 17c returning light that includes wavelengths of 405 nanometers and 635 nanometers, respectively.

In particular constructions that can be used for caries detection, the source of light can comprise a wavelength (e.g., violet light wavelength) in orange from about 360 nm to about 580 nm, or in an exemplary implementation, from about 360 m to about 420 nm, or, in a modified embodiment, monochromatic light having a wavelength of, for example, about 406 nm (e.g., visible violet wavelength) can be used.

Furthermore, continuing with exemplary implementations for caries detection, the return light can be fed through a first filter that passes radiation at wavelengths of, for example, visible red light (i.e., corresponding to the presence of caries) such as wavelengths of about 636 nm. The radiation passed by the first filter can thus be restricted at a lower end to contain mainly fluorescent radiation relatively devoid of interfering background radiation having shorter wavelengths. Moreover, the return light can also be fed through a second filter that passes radiation at wavelengths of, for example, visible green light (i.e., corresponding to the presence of healthy hard tissue) such as wavelengths of about 550 nm.

The return light exiting from the first filter (e,g., comprising visible red light corresponding to the presence of caries) can, according to one aspect of the present invention, alternatively or additionally to any other uses of the return light, be fed to an image-acquisition device (e.g., CCD or CMOS camera) for observation, analysis and/or viewing by a user. For instance, with the aid of this return light, a user may be enabled to visualize characteristics of the target surface, such as carries, and characteristics indicative of particular bacteria properties or activities such as signs of caries, including &calcifications of tooth structure caused by bacteria. Similarly, in accordance with another aspect of the invention, the return light exiting from the second filter (e.g., comprising visible green light corresponding to the presence of healthy hard tissue) can, alternatively or in addition to any other uses thereof, be fed to an image-acquisition device (e.g., CCD or CMOS camera) for observation, analysis and/or viewing (e.g., for reference or comparison purposes) by a user.

A related document, which is authored by the present Applicant, is U.S. application Ser. No. 11/203,399, filed Aug. 12, 2005 and entitled CARIES DETECTION USING TIMING DIFFERENTIALS BETWEEN EXCITATION AND RETURN PULSES (Att. Docket BI9805P; now U.S. Pat. No. 7,303,397), the entire contents of which are incorporated herein by reference. According to one embodiment which may be implemented, in whole or in part, in connection with one or more aspects of the present invention, light (e.g., violet light) in a spectral range of about 360 nm to about 580 nm, or to about 420 nm, is output as pulses of excitation light toward the target surface. In a modified implementation, the pulses of excitation light may comprise white tight. Upon receipt of the pulses of excitation light, a given carious place of a tooth will issue pulses of fluorescent radiation (e.g., visible red wavelengths).

Each pulse of return fluorescent light is received by, for example, the return-light waveguide or waveguides (e.g., the first and second return-light waveguides). These fluorescent return pulses may permit identification of different types/strains of caries-causing bacteria that return radiation of different (e.g., varying hues of red) fluorescent wavelengths. The fluorescent radiation can differ in one or more of intensity, delay and spectral distribution from radiation returned by a healthy tooth (or from radiation issued by other carious places having one or more characteristics that is/are different from the given carious place), which radiation may comprise, for example, visible green wavelengths. Thus, carious places of the tooth may, for example, appear as bright spots that stand out clearly when displayed against a dark background.

These pulses of return light can, according to certain aspects of the present invention, alternatively or in addition to any other uses of the return light, be used for automated processing (e.g., by hardware or software) and/or observation/review/analysis by a user (e.g., via feed of a light signal to the image-acquisition device). Accordingly, a condition of carious disease can be detected and viewed with a relatively high level of accuracy and reliability, at a relatively early stage. Details regarding, for example, generation of excitation light and processing of returned radiation to, for example, remove background noise and/or facilitate qualitative and quantitative detection of caries, which may be harnessed by way of one or more of automated processing and observation/review/analysis by a user according to aspects of the present invention, are described in U.S. Pat. No. 5,306,144, entitled DEVICE FOR DETECTING DENTAL CARIES, the entire contents of which are incorporated herein by reference to the extent compatible with, or modifiable by one skilled in the art to be compatible with, or to the extent not mutually exclusive with, the disclosure herein.

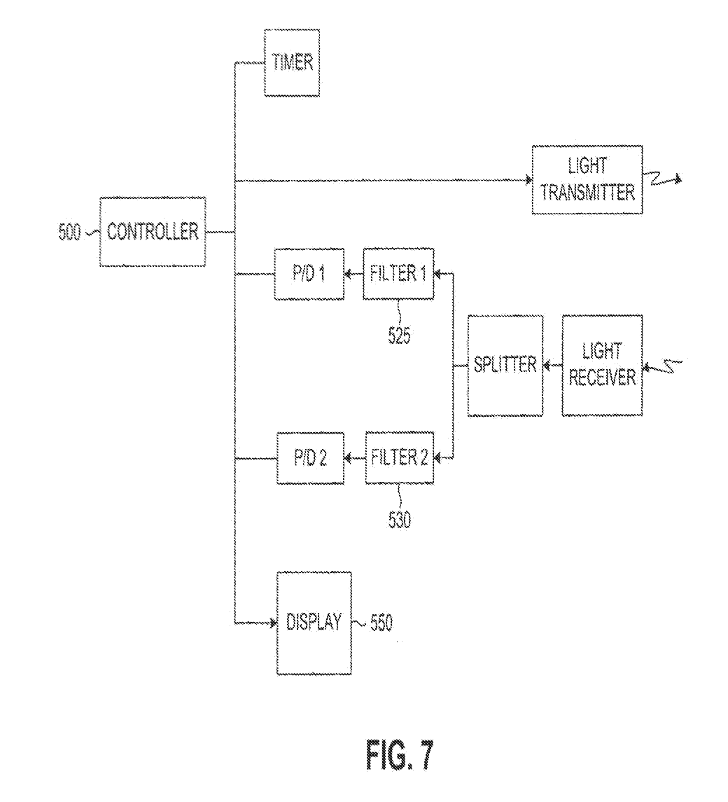

An exemplary embodiment may be implemented with, for instance, a controller as disclosed in U.S. Pat. No. 7,303,397. A block diagram of part of apparatus that may be used to detect dental caries from that patent, including a controller 500, first and second filters 525 and 530, and a display 550, is shown in FIG. 7. Returned radiation obtained from the above-mentioned first and second filters can be converted to first and second electrical signals (e.g., via one or more photo detectors), and a quotient can be obtained (e.g., automatically) by dividing the first electrical signal by the second electrical signal to provide, for example, an indication of a presence of caries. Stated otherwise, according to such an exemplary implementation, a magnitude of a green peak can be compared to a magnitude of a red peak to determine the presence and/or extent of caries. According to certain aspects of the present invention, similar protocols can be implemented, in combination with, for example, a controller, on image information, such as parts of images or entire images. For example, an image from the target surface (e.g., from one or more return-light waveguides can be passed through the first filter (e.g., on a pixel-by-pixel basis or grouped-pixel basis) to yield a first image and can be passed through the second filter (e.g., on a pixel-by-pixel basis or grouped-pixel basis) to obtain a second image. Either or both of these images may be made available for viewing on a display by a user at any stage or stages during the processing. In certain implementations a device can be configured to display a first two-dimensional image of received non-visible radiation in a form that is visible to the human eye, a second two-dimensional image of a target using visible light emitted or reflected from the target, and a third two-dimensional image of the first two-dimensional image over the second two-dimensional image wherein the first two-dimensional image appears as spots that stand out clearly against a background. Also, one or more quotients can be obtained, using, for example, the controller 500, by dividing the first image by the second image (e.g., on a pixel-by-pixel basis or grouped-pixel basis) to provide one or more images indicative of a presence of caries.

Relative time delays between a given excitation pulse and a corresponding returned pulse can be detected and processed using, for example, the controller 500. According to an implementation of a method of determining relative time delays, a running average of delays between excitation pulses and corresponding returned pulses can be maintained, in average or magnitude format, and/or in image format (e.g., on a pixel-by-pixel basis, or a grouped-pixel basis, for one or more given images). Time delays associated with an excitation pulse can be received, and the time delays between a given excitation pulse and the corresponding return pulse (e.g., time delays for various positions on the target surface, such as for various pixel positions or various grouped-pixels, or average time delays for groups of locations on the surface or for groups of pixels) can be compared with, for example, running averages of the delays or other reference values.

Time delays between transmissions of light pulses and receptions of corresponding return pulses can thus be determined. Indications may be provided, such as by the controller 500, on a display device 550 according to the time delays, as described in U.S. Pat. No. 7,303,397.

In other embodiments, using any of the above-described techniques such as multi-pixel or image protocols or variations/modifications thereof, an excitation pulse (or value/data relating thereto) may be compared with a corresponding return pulse (or value/data relating thereto) for differences in at least one of intensity, delay and spectral distribution. A given time delay (and/or another difference or other differences) between an excitation pulse and a corresponding return (e.g., fluorescence) pulse can provide, using any of the above-described techniques such as multi-pixel or image protocols or variations/modifications thereof, an indication of certain characteristics of the target surface. For example, an indication of a depth of caries can be provided, wherein a deeper (e.g., sub-surface) caries may have a greater delay and/or greater scattering than the scattering associated with surface caries or healthy tissue. In general, different lengths of excitation pulses may be able to facilitate the ascertainment of different types of information (e.g., at various points/pixels) pertaining to the target (e.g., tooth surface). A more wide-spread caries on a tooth surface may result in, for example, a return pulse having a longer fluorescence time (e.g., at certain points/pixels) when compared with less widely distributed caries. Also, a presence of different types of bacteria or conditions may be detected to an extent, for example, that different types of bacteria affect or conditions issue one or more characteristics of a return pulse differently. For instance, different types of bacteria may have different delay or fluorescence times at different points or pixels along a surface or image thereof.

As other examples of applications in the context of the present invention, visual feedback implements used with handles as described in the above-referenced U.S. Provisional Application No. 60/739,314 may be constructed and used with electromagnetic energy output devices (instead of being used with handles or in addition to such uses with handles) in similar ways (e.g., including modifications for compatibility) apparent to those skilled in the art in view of the disclosures referenced herein. As other examples, visual feedback implements used with eye treatment devices (e.g., including lasers) as described in U.S. Provisional Application No. 60/709,737 may, instead or additionally, be constructed and used with electromagnetic energy output devices in the same or similar ways (e.g., including modifications for compatibility) as may be apparent to those skilled in the art in view of the disclosures provided and referenced herein.

The above-referenced electromagnetic energy output devices, constructions and uses can be, in whole or in part, including any associated methods, modifications, combinations, permutations, and alterations of any constructions(s) or use(s) described or referenced herein or recognizable as included or includable in view of that described or referenced herein by one skilled in the art, to the extent not mutually exclusive, as described in U.S. application Ser. No. 11/033,032, filed Jan. 10, 2005 and entitled ELECTROMAGNETIC ENERGY DISTRIBUTIONS FOR ELECTROMAGNETICALLY INDUCED DISRUPTIVE CUTTING (Att. Docket BI9842P; now abandoned), U.S. application Ser. No. 11/033,043, filed Jan. 10, 2005 and entitled TISSUE REMOVER AND METHOD (Att. Docket BI9830P), U.S. application Ser. No. 11/203,400, filed Aug. 12, 2005 and entitled DUAL PULSE-WIDTH MEDICAL LASER WITH PRESETS (Att. Docket BI9808P; now abandoned), U.S. application Ser. No. 11/203,677, filed Aug. 12, 2005 and entitled LASER HANDPIECE ARCHITECTURE AND METHODS (Att. Docket BI9806P), and U.S. application Ser. No. 09/848,010, filed May 2, 2001 and entitled DERMATOLOGICAL CUTTING AND ABLATING DEVICE (Att. Docket BI9485P; now abandoned), the entire contents of all which are incorporated herein by reference. Treatments can include low-level light treatments such as described in the above-referenced U.S. Provisional Application No. 60/709,737 and U.S. Provisional Application No. 60/687,256, filed Jun. 3, 2005 and entitled TISSUE TREATMENT DEVICE AND METHOD (Att. Docket BI9846PR), the entire contents of which are incorporated herein by reference.

As an example, one implementation of a visual-feedback treatment device may be applicable for, among other things, optimizing, monitoring, or maximizing a property or condition (e.g., a cutting effect) of or in connection with use of an electromagnetic energy emitting device, such as a laser handpiece, or monitoring or detecting conditions (e.g., caries) of a target surface, such as a tooth. In any of the embodiments described or referenced herein, the electromagnetic energy (e.g., laser) output can be directed, for example, into fluid (e.g., an air and/or water spray or an atomized distribution of fluid particles from a water connection and/or a spray connection near an output end of the device) that is emitted from the visual-feedback treatment device (e.g., in the form of a handpiece) above a target surface. An apparatus including corresponding structure for directing electromagnetic energy into an atomized distribution of fluid particles above a target surface is disclosed, for example, in U.S. Pat. No. 5,574,247, the entire contents of which are incorporated herein by reference. Large amounts of electromagnetic (e.g., laser) energy, for example, can be imparted into the fluid (e.g., atomized fluid particles), which can comprise water, to thereby expand the fluid (e.g., fluid particles) and apply disruptive (e.g., mechanical) cutting forces to the target surface. During a procedure, such as an oral procedure (e.g., treatment of a periodontal pocket) where access and visibility are careful and close-up monitoring by way of a visual feedback implement of a visual-feedback treatment device of (a) interactions between the electromagnetic energy and the fluid (e.g., above the target surface), (b) cutting, ablating, treating or other impartations of disruptive surfaces to the target surface and/or (c) information on or relating to conditions of or near the target surface, can improve a quality of the procedure.

Visual-feedback treatment devices and/or periodontal probes, for example, thus can be implemented to introduce electromagnetic (e.g., laser) energy to treatment sites (e.g., diseased pockets) in a relatively controlled and precise manner to provide instantaneous visual feedback, human and/or machine readable, in accordance with an aspect of the present invention.

According to one implementation, a 400 micron flexible optical fiber (e.g., a 14 mm 400 micron periodontal tip) can be coupled to an electromagnetic energy laser) device, such as a WaterLase® or LaserSmile® device sold by Biolase Technology, Inc. of Irvine, Calif.

In the case of a diode laser, for example, an affinity for a property pigmentation can be harnessed, for example, to select (e.g., selectively destroy) certain targets (e.g., pigmented microbes) in certain instances (e.g., periodontal disease), and/or an affinity for hemoglobin can be harnessed to obtain favorable hemostasis and selectivity for vascular structures. For instance, the vascular nature of granulation tissue can allow it to be ablated with less effect on underlying tissues.

Exemplary implementations can comprise various procedures, such as laser pocket therapy (LPT), and combinations thereof, including in certain instances, alone or in combination with other steps, determinations of caries information (or, for example, in the case of laser pocket therapy, determinations of pocket depth) using a visual-feedback treatment device and/or a periodontal probe as described herein.

For example, during the selective removal of diseased lining of the periodontal pocket, iterative lasing and visual inspection of the operation site can be used to rapidly complete the procedure and to obtain a more complete and accurate removal of the diseased tissue while, for example, preserving (e.g., not removing or minimizing damage to) more of the adjacent non-diseased tissue. The visual-feedback treatment device can be used at a pre-treatment stage to facilitate implementation of a patient diagnosis (e.g., including a full mouth probing of six sites per tooth, radiographs, thorough root planing and scaling), followed by, for example, the performance of laser pocket therapy on areas that are, for example, 5 mm or greater. The treated areas can then be evaluated and retreated with the visual-feedback treatment device if, for example, the pockets have remained greater than 5 mm following a period of time. Similar evaluations and retreatments can be repeated, for example, every three months until, for example, pockets are less than 5 mm or improvement no longer continues.

When using a fiber that can leech energy from the sides, such as that described above in combination with a diode LaserSmile® device or as described, for example, in U.S. application Ser. No. 11/033,441, filed Jan. 10, 2005 and entitled MODIFIED-OUTPUT FIBER OPTIC TIPS (Att. Docket BI9827P; now U.S. Pat. No. 7,620,290), the entire contents of which are incorporated herein by reference, treatment can in certain embodiments be performed using a sweeping motion. According to an exemplary application, the fiber can be inserted to a depth of a pocket and swept across the entire pocket, using the visual feedback implement of the visual-feedback treatment device for precision and speed. Local anesthesia can be provided by way of, for example, a diode.

When using a WaterLase®, such as a WaterLase® YSGG laser, that has an affinity for water, microbes, which predominantly comprise water, can be instantly destroyed even beyond the zone of ablation when electromagnetic (e.g., laser) energy is introduced. When using fiber tips that leech energy from the end of the tip, and not from the side, the technique for introducing electromagnetic (e.g., laser) energy to, for example, a pocket, can be similar in some instances to probing. In such an example, with the laser on, the fiber can be pumped up and down from the crest of the gingiva, to the bottom of the pocket. As this technique is performed, the tip can be moved along the length of the targeted area (e.g., pocket) with overlapping strokes as if step probing.

In accordance with the just-described example and in any other embodiments described, referenced, or suggested herein, the following disclosure may apply.

The use of electromagnetic (e.g., laser) energy in conjunction with collection of image data in a procedure, such as, for example, a treatment performed inside of a diseased pocket formed adjacent to a tooth, can be utilized to visualize tissue for removal, modification, and/or other treatment, either simultaneously or sequentially. Visualization of, for example, inflamed, infected and/or necrosed tissue for adequate removal can be clinically advantageous.

The monitoring, treatment (e.g., repair) or other collection of data regarding tissue can be monitored through, for example, any of the optical feedbacks described or referenced herein. For example one method of monitoring tissue regeneration by type can be through Optical Coherence Tomography. In periodontics, for example, it can be very important to monitor the repair of ligament fibroblasts for re-attachment and also the osseous tissue lost during progression of disease. The camera can also utilize Optical Doppler Tomography to detect changes in blood flow and circulation. The same apparatus can be utilized to conduct surgery and/or treat and visualize various target sites in connection with various procedures (e.g., such as to visualize the inside a root canal that has been affected by bacteria or any lumen that is accessible to a fiber optic that may be utilized to treat or conduct surgery). :Inside a root canal it can be important to visualize the presence of necrosed or bacterial affected (infected) tissue as well as the condition of the root canal wall or any lateral canals. The camera may also provide, for example, direction inside on how to guide the surgical/therapeutic beam through a curved canal.

In various medical procedure (e.g., dental, hard tissue) contexts, the visual-feedback treatment device of the present invention can be implemented to facilitate, in whole or in part, any of the following procedures: class I, II, III, IV, and V cavity preparation; caries removal; hard tissue surface roughening or etching; enameloplasty, excavation of pits and fissures for placement of sealants; osseous crown lengthening; cutting, shaving, contouring and resection of oral osseous tissues (bone); osteoplasty and osseous recontouring (removal of bone to correct osseous defects and create physiologic osseous contours); ostectomy (resection of bone to restore bony architecture, resection of bone for grafting, etc); cutting bone to prepare a window access to the apex (apices) of the root(s); apicoectomy—amputation of the root end; root end preparation for retrofill amalgam or composite; tooth preparation to obtain access to root canal; root canal preparation including enlargement; and root canal debridement and cleaning.

According to various other medical areas (e.g., dental, soft tissue and other) contexts, the visual-feedback treatment device of the present invention can be implemented to facilitate, in whole or in part, any of the following procedures: incision, excision, vaporization, ablation and coagulation of oral soft tissues; excisional and incisional biopsies exposure of unerupted teeth; fibroma removal; flap preparation—incision of soft tissue to prepare a flap and expose the bone; frenectomy and frenotomy; gingival troughing for crown impressions; gingivectomy; hemostasis; implant recovery; incision and drainage of abscesses; operculectomy; pulpotomy; pulp extirpation; pulpotomy as an adjunct to root canal therapy; root canal debridement and cleaning; removal of pathological tissues from around the apex; soft tissue crown lengthening; sulcular debridement; treatment of canker sores, herpetic and aphthous ulcers of the oral mucosa; vestibuloplasty; flap preparation—incision of soft tissue to prepare a flap and expose unerupted teeth (hard and soft tissue impactions); removal of granulation tissue from bony defects; laser soft tissue curettage; and specialty procedures, such as osseous crown lengthening, periodontal therapy (e.g., including laser curettage, sulcular debridement, removal of pathological tissue, and the like), oral surgery and implant applications (e.g., including harvesting block graft tissue or creating a pilot hole for an implant), and endo (e.g., including pulp therapy, canal access, shaping and debridement).

While the description above refers to particular embodiments of the present invention, it will be understood that many modifications may be made without departing from the spirit thereof. Aspects of the invention may have combinations of the above-described embodiments although these combinations may not be explicitly described. Any accompanying additional disclosure in claims format is intended to cover such embodiments as would fall within the true scope and spirit of the present invention.

What is claimed is:

1. An electromagnetic energy output device, comprising:
a trunk assembly constructed to output electromagnetic energy;
an output fiber tip coupled to receive electromagnetic energy from the trunk assembly; and
an image acquisition device.

2. The electromagnetic energy output device as set forth in claim 1, wherein the electromagnetic energy output device is a handpiece.

3. The electromagnetic energy output device as set forth in claim 2, wherein the electromagnetic energy output device is a laser dental handpiece.

4. The electromagnetic energy output device as set forth in claim 2, wherein the image acquisition device is a camera.

5. The electromagnetic energy output device as set forth in claim 4, wherein the image acquisition device is a digital camera.

6. The electromagnetic energy output device as set forth in claim 4, wherein the camera is part of the trunk assembly.

7. The electromagnetic energy output device as set forth in claim 4, and further comprising one or more electromagnetic energy waveguides for emitting illumination light for enhancing user viewability of a target surface.

8. The electromagnetic energy output device as set forth in claim 4, and further comprising one or more electromagnetic energy waveguides for emitting excitation light and for receiving and processing return excitation light.

9. The electromagnetic energy output device as set forth in claim 8, wherein the camera is part of the trunk assembly.

10. The electromagnetic energy output device as set forth in claim 4, and further comprising one or more electromagnetic energy waveguides for emitting illumination and excitation light for purposes of enhancing user viewability of a target surface, and for receiving and digitally processing return excitation light.

11. An apparatus in the form of an electromagnetic energy output device, the apparatus comprising:
a power supply, a pumping source, and up network capable of driving the pumping source with an output pulse having a first duration, an output pulse having a second, greater duration, an output pulse having a third duration, and at least one or more output pulses having other durations; and
an excitation source, which is configured to direct electromagnetic energy into a volume, wherein the excitation source outputs the electromagnetic energy in a form of at least one output pulse, whereby placement of the volume adjacent to or over a target during use facilitates impartation of cutting or ablating forces to the target.

12. The apparatus as set forth in claim 11, the apparatus further comprising:
an emitting end; and
a fluid output oriented and configured to place fluid into a volume in proximity to the emitting end.

13. The apparatus as set forth in claim 12, wherein the excitation source is configured to direct electromagnetic energy into the volume in proximity to the emitting end, wherein the excitation source outputs the electromagnetic energy in a form of at least one output pulse, having a plurality of high-intensity leading micropulses, that imparts relatively large amounts of energy into at least part of the fluid in the volume, the relatively large amounts of energy imparted into the fluid being sufficient to cause the fluid to expand whereby placement of the volume adjacent to or over a target during use facilitates impartation of cutting or ablating forces to the target.

14. The apparatus as set forth in claim 11, wherein:
the power supply is a single power supply capable of supplying a first high voltage output at a first voltage level, a second high voltage output at a second voltage level, and at least two more high voltage outputs at respective additional voltage levels;
the pulse-forming network comprises first, second, and at least two more pulse-forming networks;
the first pulse-forming network is capable of receiving the first high voltage output, the first pulse-forming network further being capable of driving the excitation source with a first signal;
the second pulse-forming network is capable of receiving the second high voltage output, the second pulse-forming network further being capable of driving the excitation source with a signal differing from the first signal in one or more of duration and magnitude; and
the at least two more pulse-forming networks are capable of receiving the additional high voltage outputs and driving the excitation source with signals differing from the first signal in one or more of duration and magnitude.

15. The apparatus as set forth in claim 14, wherein:
the first pulse-forming network has a capacitor of about 30-70 μF and an inductor of about 30-70 μH; and
the second pulse-forming network has a capacitor of about 300-600 μF and an inductor of about 800-1200 μH.

16. A method of using a power supply according to claim 11 to generate three or more pulse outputs for an electromagnetic energy output device, the method comprising:
providing from the power supply a first high voltage output capable of driving a first pulse-forming network that has a capacitor of about 30-70 μF and an inductor of about 30-70 μH and is configured to generate a first pulse output;
providing from the power supply a second high voltage output capable of driving a second pulse-forming network that has a capacitor of about 300-600 μF and an inductor of about 800-1200 μH and is configured to generate a second pulse output; and
providing from the power supply at least two more high voltage outputs capable of driving respective additional pulse-forming networks.

17. A method of imparting disruptive forces onto a target surface, the method comprising:
providing a circuit having a high voltage power supply according to claim 1 capable of generating a first pulse output, a second pulse output having a duration greater than that of the first pulse output, a third pulse output, and at least one more pulse output;
placing fluid into a volume above the target surface; and
directing energy corresponding to one or more of the pulse outputs into the volume, whereby energy from at least one of the outputs comprises a plurality of high-intensity leading micropulses that impart relatively large amounts of energy into at least part of the fluid in the volume causing the fluid to expand, and whereby disruptive cutting or ablating forces are imparted onto the target surface.

18. A method of using a power supply according to claim 11 to generate three or more pulse outputs for an electromagnetic energy output apparatus, the method comprising:
providing more than three high voltage outputs from the power supply; and
providing more than three pulse-forming networks having capacitors of about 30-70 μF and 300-600 μF and inductors of about 30-70 μH and 800-1200 μH, wherein the plurality of high voltage outputs drives the plurality of pulse-forming networks with signals differing in one or more of duration and magnitude.

19. A method of using the apparatus according to claim 11 for imparting disruptive forces to a target, the method comprising:
providing a first pulse output, a second pulse output capable of outputting a pulse with a greater duration than a pulse outputted by the first pulse output, a third puke output, and at least one more pulse output;
placing fluid into a zone above the target; and
directing energy corresponding to one or more of the pulse outputs into the zone, whereby high-intensity leading micropulses of energy from one or more of the pulse outputs is imparted to at least part of the fluid in the zone causing the fluid to expand, and whereby disruptive cutting or ablating forces are imparted to the target.

20. The electromagnetic energy output device as set forth in claim 1, and further comprising an image acquisition fitting for routing images acquired at or in a vicinity of the distal end of the electromagnetic energy output device.
